(19) United States
(12) Patent Application Publication
(10) Pub. No.: US 2023/0293425 A1
(43) Pub. Date: Sep. 21, 2023

(54) ANTIMICROBIAL TOPICAL COMPOSITIONS CONTAINING MANUKA OIL

(71) Applicant: Manuka Therapeutics Ltd., Auckland (NZ)

(72) Inventors: Robert F. Gilmour, Woolloomooloo (AU); Stuart H. Cairns, Auckland (NZ); Christine J. Hames, Auckland (NZ)

(21) Appl. No.: 18/187,132

(22) Filed: Mar. 21, 2023

Related U.S. Application Data
(63) Continuation of application No. 16/986,100, filed on Aug. 5, 2020, now abandoned.
(60) Provisional application No. 62/882,672, filed on Aug. 5, 2019.

(51) Int. Cl. A61K 9/00; A61K 36/61; A61K 31/23; A61K 36/71

(52) U.S. Cl. CPC A61K 9/0014; A61K 36/61; A61K 31/23; A61K 36/71; A61K 9/06

(57) ABSTRACT

A topical composition for treatment of a skin condition comprising a mixture of Leptospermum scoparium oil (manuka oil), Nigella sativa seed oil (black seed oil), and a carrier composition.

### RELATED APPLICATIONS

This application is a continuation of U.S. Utility application Ser. No. 16/986,100, filed on Aug. 5, 2020 and claims priority to U.S. Provisional Application No. 62/882,672 filed on Aug. 5, 2019.

### FIELD OF THE INVENTION

The application relates to topical compositions for the treatment of damaged skin in mammals, including humans.

### BACKGROUND

Various ailments, diseases, or injuries can cause skin conditions. These causes include, but are not limited to, physical injury or trauma such as lacerations, chapping, burns, blistering, and the like; diseases; physical disorders such as allergic reactions, dryness, acne, or the like; microbes including bacteria, fungi, viruses, parasites, and the like; or other causes. Damaged skin, particularly those resulting in an open wound, may be susceptible to microbial infection, which can impede or inhibit the body's natural ability to heal. Many options and approaches have been used for treating damaged skin designed to protect or promote skin healing.

### SUMMARY

In one embodiment in accordance with this disclosure, a topical composition for treatment of a variety of skin conditions includes a mixture of Leptospermum scoparium oil (manuka oil) and Nigella sativa seed oil (black seed oil), optionally ethylhexyl palmitate, and a carrier composition. In some embodiments, the combination of manuka oil and black seed oil provides a synergistic effect that provides excellent and sustained antimicrobial activity as compared to other topical compositions that include either of the oils independently. The topical compositions disclosed may be applied to damaged skin to reduce or prevent microbial growth and improve the rate of healing of the damaged area.

In an embodiment in accordance with this disclosure, a topical composition for treatment of a skin condition comprising a mixture of Leptospermum scoparium oil (manuka oil), Nigella sativa seed oil (black seed oil), ethylhexyl palmitate, and a carrier composition.

In an embodiment in accordance with this disclosure, a topical composition for treatment of a skin condition in the form of a topical gel. The topical composition comprising a mixture of Leptospermum scoparium oil (manuka oil), Nigella sativa seed oil (black seed oil), and a carrier composition comprising predominately water.

In another embodiment in accordance with this disclosure, a method for treating a skin condition comprising applying a topical composition to an area containing a skin condition, the topical composition comprising Leptospermum scoparium oil (manuka oil), Nigella sativa seed oil (black seed oil), ethylhexyl palmitate, and a carrier composition, wherein the manuka oil, the black seed oil, and the ethylhexyl palmitate are mixed in the carrier composition.

In another embodiment in accordance with this disclosure, a method for manufacturing a topical composition used to treat a skin condition comprising mixing Leptospermum scoparium oil (manuka oil), Nigella sativa seed oil (black seed oil), and other ingredients into a carrier composition.

The above summary is not intended to describe each disclosed embodiment or every implementation of the embodiments disclosed herein. The description that follows more particularly exemplifies illustrative examples. In several places throughout the application, guidance is provided through lists of examples, which can be used in various combinations. In each instance, the recited list serves only as a representative group and should not be interpreted as an exclusive list.

### Definitions

As used herein, “a,” “an,” “the,” “at least one,” and “one or more” are used interchangeably. Thus, for example, a topical composition that comprises “a” natural wax can be interpreted to mean that the topical composition includes “one or more” natural waxes.

As used herein, the term “consists substantially of” means that at least 95 percent by weight (wt. %) the respective material is made of the recited component. For example, a carrier composition that consists substantially of vegan materials means that at least 95 wt. % of the carrier composition is made of vegan materials.

As used herein, the term “consists essentially of” means that the respective material is formulated of the recited components, but also may include other contaminates that do not materially affect the physical or chemical properties of the material. In some embodiments, a composition that “consists essentially of” a list of materials may include less than 0.5 wt. % other components.

As used herein, the term “comprises” and variations thereof do not have a limiting meaning where these terms appear in the description and claims.

As used herein, the term “essential oil,” “plant oil,” or “seed oil,” refers to a natural, plant-based oil. Unless indicated otherwise, such oils may derived from a cold press process or a steam distillation. The method of producing such oils (e.g., steam distillation versus cold press) may alter the compositional makeup and physical and medicinal properties of the oil.

As used herein, the terms “mixture” or “mixing” refers to a physical mixture or physically mixing (e.g., blending) two or more components together. Unless otherwise indicated, a mixture includes emulsions, heterogeneous mixtures, and homogeneous mixtures.

As used herein, the term “natural” refers to an excipient derived from a renewable source such as plants, insects, or animal-based sources, as opposed to synthetic or petroleum-based sources. For example, the phrase “natural waxes” may include waxes derived from plants, insects (e.g., bees), or animals. An excipient may still be considered “natural” even though the excipient may have undergone a chemical or physical extraction process. For example, essential oils are typically extracted from plants through a distillation process. Essential oils are still considered natural even though processing has occurred to isolate and purify the oil.

As used herein, the terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, the term “topical composition” refers to compositions designed to be applied to the skin of a patient and is not indented for internal consumption or use. A topical composition may include transdermal excipients (e.g., those that penetrate or pass through the layers of the epidermis) and may be applied to portions of the skin that include an open wound (e.g., laceration, abrasion, surgical incision, and the like). Topical compositions may include, but are not limited to balms, creams, gels, lotions, ointments, solutions, sprays, or the like. Topical compositions may be applied directly to the skin of a patient (e.g., direct application of a balm) or indirectly to the skin of a patient (e.g., application to a wound dressing that is applied to the skin).

Also herein, the recitations of numerical ranges by endpoints include all numbers subsumed within that range (e.g., 1 to 5 includes 1, 1.5, 2, 2.75, 3, 3.80, 4, 5, etc.). Furthermore, disclosure of a range includes disclosure of all sub-ranges included within the broader range (e.g., 1 to 5 discloses 1 to 4, 1.5 to 4.5, 4 to 5, etc.).

### DETAILED DESCRIPTION

The embodiments described in this disclosure provide a topical composition for the treatment of various skin conditions and method of producing and using such compositions. Various embodiments are effective at treating many forms of skin conditions including damage due to abrasions, disease, exposure to toxins and radiation, immune responses, injury, medical procedures, microbes, trauma, and the like.

In some embodiments, the disclosed topical compositions include a unique mixture of Leptospermum scoparium oil (manuka oil), Nigella sativa seed oil (black seed oil), optionally ethylhexyl palmitate, and a carrier composition. As discussed further below, the inclusion of both manuka oil and black seed oil provide synergistic effects that increase the antimicrobial activity and duration of effectiveness of the topical composition compared to the use of either oil individually. The combination also provides additional advantages as discussed further below. The inclusion of ethylhexyl palmitate enhances the physical properties of the topical composition, particularly in a topical balm, that helps retain the manuka and black seed oils against the user's skin while simultaneously promoting hydration and skin healing conditions. The topical compositions disclosed provide a protective barrier that enhances healing for a variety of skin conditions as well as enhances the tactile and wearability attributes of the topical composition.

In accordance with various embodiments, some topical compositions disclosed include a mixture of Leptospermum scoparium oil (manuka oil) and Nigella sativa seed oil (black seed oil). Manuka oil may be extracted from the leaves of a Leptospermum scoparium plant using a steam distillation process. Leptospermum is the botanical name given to New Zealand's Manuka tree. There are two species of Leptospermum in New Zealand, distinguishable by white or red pedals they produce. Leptospermum scoparium or L. scoparium refers to the white Manuka tree. While the Manuka tree is sometimes referred to as the New Zealand Tea Tree, oil extracted from the Manuka tree should not be confused with “tea tree oil.” In contrast to manuka oil, conventional tea tree oil is derived from the plant Melaleuca alternifolia, commonly found in Australia. Both the Melaleuca alternifolia and Leptospermum scoparium belong to the myrtle family Myrtaceae commonly known as tea trees however, the oils extracted from each have comparatively different properties and chemical compositions.

Manuka oil includes three major groups of compounds including monoterpenes, sesquiterpenes, and β-triketones. Monoterpenes can include, for example, α- and β-pinene. Sesquiterpenes can include, for example, α- and β-eudesmol and β-caryophyllene. β-triketones include, for example, flavesone, iso-leptospermone, and leptospermone. Without being bound to a specific theory, the β-triketones are believed to contribute to the exceptional antimicrobial properties of manuka oil and provide the synergistic effect when combined with a composition containing black seed oil.

The amount β-triketones contained within manuka oil extract from the Manuka tree can depend on the region within New Zealand in which the Manuka tree is harvested. For example, Manuka trees harvested from the East Cape region of New Zealand contain the highest levels of β-triketones compared to any other region, presumably due to the unique growing conditions of the region. The levels of β-triketones within the manuka oil extracted from Manuka trees grown the East Cape region of New Zealand can be greater than 20 weight percent (wt. %), and in some examples greater than 30 wt. %, as compared to Manuka trees harvested from other regions of New Zealand which are typically far less than 20 wt. % and most commonly less than 5 wt. %. In some embodiments, the manuka oil used in the disclosed topical compositions may be derived from Manuka tress sourced from the East Cape region of New Zealand.

In preferred embodiments, the topical compositions disclosed include manuka oil having a relatively high concentration of β-triketones. In some examples, the manuka oil may include at least 10 wt. % β-triketones, at least 25 wt. % β-triketones, at least 30 wt. % β-triketones, and most preferably at least 40 wt. % β-triketones. In some examples, a double steam distillation process may be used to extract and concentrate the manuka oil to increase the relative concentration of β-triketones within the oil. Sourcing the Manuka oil from Manuka trees harvested from the East Cape region of New Zealand provides a rich concentration of β-triketones within the extracted oil that likewise preservers the other components present in the extracted oil.

Manuka oil is commercially available from Tairawhiti Pharmaceuticals Ltd. of East Cape, New Zealand and New Zealand Manuka Bioactives of Opotiki, New Zealand. New Zealand Manuka Bioactives offers different grades of manuka oil with different levels of β-triketones including, for example, MβTK™ 5+, MβTK™ 10+, MβTK™ 25+, MβTK™ 30+, and MβTK™ 40+. New Zealand Manuka Bioactives characterizes its MβTK™ 25+ and higher grades as being sourced from the Manuka trees harvested from the East Cape region of New Zealand.

As discussed further below, manuka oil has a high antimicrobial activity. In some embodiments, topical compositions disclosed that include manuka oil may be useful in preventing of inhibiting microbial growth associated with skin abrasions, acne, lacerations, bacterial infections, fungal infections, viral infections, and the like. Manuka oil exhibits good antimicrobial activity against a variety of microbes (e.g., bacteria, fungi, viruses, and the like) including, but not limited to, Bacillus subtilis, Candida albicans, Clostridium species, Fusarium circinatum, gram positive bacteria, Herpes simplex-1 and -2 virus, Micrococcus luteus, Mycobacterium phlei, Sarcina lutea, Staphylococcus species, Legionella pneumophila, Listeria monocytogenes, and others.

In some embodiments, the topical compositions disclosed include black seed oil. Black seed oil may be extracted from the seeds of a Nigella sativa plant, which is a small plant with pale purple, blue, or white flowers that grows in Eastern Europe, Western Asia, and the Middle East. In some embodiments, the black seed oil may be extracted from the seeds using a cold press process.

Black seed oil extracted from the seeds of a Nigella sativa plant has a high p-cymene and thymoquinone content. Without being bound to a specific scientific theory, it is believed that the thymoquinone contributes to antimicrobial and anti-inflammatory properties of the black seed oil and provides the synergistic effect with manuka oil. Black seed oil may be used on lacerated or damaged skin to help prevent or inhibit bacterial growth and thereby promote skin healing. The anti-inflammatory properties of black seed oil may also help to soothe irritation and reduce swelling or redness of inflamed skin. In topical compositions, the black seed oil may be useful for treating a large number of skin conditions including for example, acne, eczema, and the like.

The combination of black seed oil and manuka oil produce several surprising synergistic effects when mixed together in a topical composition. For example, the mixture of black seed oil and manuka oil increases the antimicrobial activity of the composition compared to either of the two oils individually. Additionally, the mixture of black seed oil and manuka oil shows a broader spectrum of antimicrobial activity and improved activity for longer periods of time. Without being bound to a specific scientific theory, it is believed that the mixture of black seed oil and manuka oil may not only inhibit the growth of microbes on the surface of the skin but also impede the chance of resistance mutation within the microbe. This may be due in part to the complexity and diversity of the various components contained within both oil extracts making it difficult for mutations of such microbes to develop resistance. Accordingly, the topical compositions disclosed not only remain more effective for a longer period of time with a single application, but also remain effective during the healing process even after multiple applications over an extended period.

In some embodiments, the ratio of manuka oil to black seed oil may be about 1:1 to about 10:1 by weight. In preferred embodiments, the ratio of manuka oil to black seed oil may be adjusted to include a higher weight percent of manuka oil compared to black seed oil (e.g., about 4:1).

In some embodiments, the topical compositions disclosed, particularly topical balms, also include ethylhexyl palmitate. Ethylhexyl palmitate is a natural, organic alternative to silicone-based materials and acts as a non-occlusive emollient that provides a dry-slip, silky feel reminiscent of silicone-based products. As a non-occlusive emollient, the ethylhexyl palmitate does not inhibit the passage of air and oxygen through the topical composition to promote skin-healing conditions. The ethylhexyl palmitate may be particularly useful for skin conditions that relate to dry skin such as, for example, eczema, psoriasis, and other inflammatory skin condition. The ethylhexyl palmitate may keep the skin moist and supple by reducing water loss from the epidermis, which in turn can promote skin healing. Additionally, ethylhexyl palmitate may act as a lubricant within the topical composition to help reduce friction between the skin and external contact to prevent chafing and promote healing. The ethylhexyl palmitate may also function as a solvent to help dissolve some of the other ingredients to allow for a more homogeneous mixture.

The ethylhexyl palmitate also provides a synergistic effect when formulated with manuka oil and black seed oil. Without being bound to a specific scientific theory, ethylhexyl palmitate is believed to reduce the volatility of the manuka oil, black seed oil, and other material to help retain such materials within the topical composition for an extended period of time while also promoting an optimized environment for skin healing. This in turn allows the manuka and black seed oils to be added to the topical compositions in lower concentrations while still providing excellent antimicrobial activity for an extended period of time. The ethylhexyl palmitate may also allow the topical composition to have a longer storage shelf life.

In some embodiments, the topical compositions disclosed may include any suitable ratio or combination of the excipients (e.g., manuka oil, black seed oil, and ethylhexyl palmitate) disclosed above. The topical composition may include less than about 1 wt. % of manuka and black seed oil (e.g., about 0.5 wt. % in total) and about 1 wt. % ethylhexyl palmitate based on the total weight of the topical composition with the balance provided by the carrier composition. The amount of materials may be more or less to obtain the desired characteristics depending on the carrier material used.

The topical compositions disclosed also include a carrier composition. The carrier composition may include natural or vegan materials. For example, with modern sophisticated consumers, there exists a strong desire and preference to use natural, environmentally friendly materials rather than synthetic or petroleum based materials. Accordingly, in preferred embodiments, the carrier composition may consist substantially of or may consist essentially of natural or vegan based materials. Likewise, it may be desirable for the topical composition to consist substantially of or consist essentially of natural or vegan based materials.

Additionally or alternatively, the carrier composition may include predominately water (e.g., more than 75% of the composition is water). Water based compositions may be particularly useful in the formation of a topical gel, allowing for the majority of the composition to evaporate after application. Such topical gels may be used as a blemish gel.

The topical composition may be formulated into any suitable type of vehicle including, for example, balms, creams, gels, lotions, ointments, solutions, sprays, or the like. In preferred embodiments, the topical composition is formulated into a topical balm or a topical gel. Topical balms are composed of predominately non-volatile materials. The vast majority of the materials in a balm remain as a solid film on the skin. In contrast, gels are predominantly composed of a volatile carrier (e.g., water or alcohol) that evaporates upon application. The below examples provide representative topical balms and topical gels containing the manuka oil and black seed oil compositions. However, other base balm or gel compositions that are not described herein may also be used provided they include the disclose manuka oil based or manuka oil based and black seed compositions described herein.

The carrier composition may include one or more natural waxes that serve as a vehicle for the composition. Natural waxes provide a “green” alternative to synthetic or petroleum based derivatives. Natural waxes derived from plant-based materials or insect-based materials (e.g., bees) provide vegan-friendly source materials. The one or more natural waxes may act as emulsifiers that help keep the mixture of oils and other excipients from separating into the liquid and oil components. The waxes may also contribute to the sensory characteristics (e.g., touch and feel) of the topical composition. Suitable natural waxes may include, but are not limited to, beeswax, candelilla wax, carnauba wax, jasmine wax, jojoba wax, laurel wax, momosa wax, myrica fruit wax, orange wax, phyto wax, rapeseed wax, Rhus verniciflua peel wax (e.g., berry wax), Rhus succedanea fruit wax (e.g., Japan wax), rice bran wax, rose wax, soy wax, sunflower seed wax, tea wax, mixtures thereof and the like.

In preferred examples, the topical compositions include one or more of beeswax, Rhus succedanea fruit wax, and Rhus verniciflua peel wax. Beeswax, sometimes referred to as cera alba, is secreted by bees and can be sourced from a variety of places. Beeswax exhibits a relatively high melting point (e.g., about 61-65° C.) that remains workable at ambient to body temperatures. Beeswax has a silky texture and does not clog pores, both of which are desirable for skin applications. The beeswax may help to form a protective layer over skin to help prevent loss of moisture and help protect the skin from environmental conditions and external moisture. Beeswax also has the ability to act as an emulsifier in the topical composition.

Rhus succedanea fruit wax is obtained from the berries of the Rhus succedanea plant, which grows in Japan and China. Sometimes referred to as Japan wax, Rhus succedanea fruit wax has a relatively low melting point (e.g., about 45-55° C.) and acts as an emulsifier in the topical formulation to help prevent the separation of the oils and other excipients. Rhus succedanea fruit wax may also help to increase the plasticity of the topical composition to provide a semi-solid consistency that still allows for smooth and effective application of the composition as a topical balm.

Rhus verniciflua peel wax, also referred to as berry wax, is a soft wax derived from the peel of the fruit of the Rhus verniciflua plant. Berry wax has a relatively low melting point (e.g., about 48-55° C.). The wax is used to give a soft consistency that provides a cooling sensation with a silky feel and very desirable sensory characteristics when applied to the skin. Like Rhus succedanea fruit wax, Rhus verniciflua peel wax may also help to increase the plasticity of the topical composition to provide a semi-solid consistency that still allows for smooth and effective application of the composition as a topical balm.

The carrier composition may also include other optional excipients including, but not limited to, antioxidants, emollients, moisturizers, surfactants, lubricants, natural oils, nutrients or vitamins, and the like. In many examples, a particular excipient may serve multiple functions within the topical composition. For example, tocopherol is both an antioxidant and vitamin that serves as a nutrient for the skin.

Optional excipients may include one or more antioxidants including, but are not limited to, tocopherol (vitamin E) and ascorbyl palmitate (vitamin C ester). Tocopherol is typically derived from vegetable oils and possesses exceptional skin protection properties. For example, tocopherol may be used to absorb UV rays and prevent UV induced free radical damage to the skin as well as prevent other free radical induced damage. Tocopherol has anti-inflammatory properties and helps moisturize the skin. Ascorbyl palmitate is a fat-soluble derivative of ascorbic acid that acts as a free radical scavenger. Unlike the water soluble form (e.g., ascorbic acid), ascorbyl palmitate is able to enter the lipid cell membrane of the skin. Ascorbyl palmitate also supports immune cell activity and helps with the formation and maintenance of collagen within the skin. Both tocopherol and ascorbyl palmitate may be produced from natural and vegan-based sources. In preferred embodiments, the topical composition includes tocopherol, ascorbyl palmitate, or both.

Other useful optional excipients include nutrients and vitamins. In some embodiments, the carrier composition may include one or more vitamins A, C, D, E, or K. Preferred vitamins include vitamin A (e.g., retinol or retinol ester), vitamin C (e.g., ascorbyl palmitate or ascorbic acid), and vitamin E (tocopherol).

In some embodiments, the topical composition may also include at least vitamin A (e.g., retinol or retinol ester). The inclusion of retinol in the topical composition in conjunction with the other materials disclosed above may be useful for the treatment of acne.

Other useful optional excipients include natural oils, such as one or more plant oils, seed oils, essential oils, or the like. Natural oils may serve multiple functions within the topical composition such as acting as both a lubricant and emollient. Suitable natural oils may include, for example, Ricinus communis seed oil (e.g., castor oil) and Simmondsia chinensis seed oil (e.g., jojoba oil). Castor oil is a plant oil derived from the seeds of a Ricinus communis plant. Castor oil includes ricinoleic acid, which is a monounsaturated fatty acid that acts as a humectant. Castor oil helps moisturize the skin by preventing water loss, which in turn may help promote skin healing. Castor oil also has anti-inflammatory properties, antimicrobial activity, and can alleviate pain in some instances. Jojoba oil is a plant oil derived from the seeds of a Simmondsia chinensis plant. Jojoba oil acts as a moisturizer and emollient agent to improve the skin elasticity and suppleness, containing natural tocopherol to minimize oxidation. Jojoba oil behaves similarly to the natural oils of the skin and can penetrate the layers of the skin quickly to help promote healing without clogging pores or leaving a greasy feel to the skin. Because both castor and jojoba oil are derived from plants, they provide vegan-based materials for the topical composition. In preferred embodiments, the topical composition includes jojoba oil, castor oil, or both.

In some embodiments, the carrier composition may include one or more pre-formulated carrier compositions prepared by third party manufacturers. For example, the carrier composition may include one or more Kahl Jellies (e.g., VegoJelly 7036Plus), Kahl Bases (e.g., Natural Lip Care Base 7704), or Kahl Specialty Waxes (e.g., Veggiesoft Complex 6422), or similar products each available from Kahl GmbH & Co. KG of Germany Such materials may include a blend of different excipients. For example, VegoJelly 7036Plus is listed as including Ricinus communis seed oil, Rhus verniciflua peel wax, Rhus succedanea fruit wax, ascorbyl palmitate, and tocopherol.

In some embodiments, the topical compositions may include one or more pharmaceutically active excipients. Such pharmaceutically active excipients may be applied tropically to treat a topical condition of the skin or be configured to be transmitted transdermally (e.g., transmitted into various layers of the skin or through the layers into the deep tissue or blood stream of the patient). In some examples, the pharmaceutically active excipient may be used to treat skin ailments such as, but not limited to, acne, bacterial infections, eczema, fungal infections, psoriasis, rashes, viral infections, or the like. In some such examples, the carrier composition may be formulated using pharmaceutically acceptable and compatible excipients. The described characteristics of manuka oil, black seed oil, and ethylhexyl palmitate may help to improve the efficacy of such pharmaceutically active excipients, limit the side effects of such pharmaceutically active excipients, or both.

As discussed above, in preferred embodiments, the topical composition consists substantially or essentially of natural or vegan based materials. However, several pharmaceutically active excipients may not be considered natural or vegan based excipients. In such embodiments, the topical composition serving as the vehicle for the pharmaceutically active excipients may nevertheless be characterized as consisting substantially or essentially of natural or vegan based materials.

The topical compositions disclosed may be used to treat one or more skin conditions including, but not limited to, inhibiting microbial growth on skin, treating skin lacerations or abrasions, treating acne, reducing the chance of infection after medical procedures such as those where the epidermis is breached by a needle, cannula, scalpel, or the like. The topical compositions may be applied directly to the afflicted area containing the skin condition. For example, the patient's skin may be initially cleaned around the site of the condition. The topical composition, such as a topical balm, may be applied liberally to the site and allowed to absorb into the skin. If additional protection is desired, a wound dressing or bandage may be applied over the topical composition, though the topical composition may also be used without such wound dressings. Additionally, or alternatively, the topical composition may be applied to a wound dressing or bandage and then applied to the site containing the skin condition.

### EXAMPLES

The following examples are offered to aid in understanding of the above embodiments and are not to be construed as limiting the scope thereof.

### Example 1—Topical Balm

A mixture of the following excipients were prepared to produce a topical balm.

| | Amount |
|---|---|
| Component | (wt. %) |
| Manuka oil | 0.4 |
| Black seed oil | 0.1 |
| Ethylhexyl palmitate | 1.0 |
| Kahl VegoJelly 7036Plus (carrier, mixture of Ricinus Communis | 98.3 |
| seed oil, Rhus Verniciflua peel wax, Rhus Succedanea fruit wax, | |
| ascorbyl palmitate, and tocopherol). | |
| Jojoba oil | 0.1 |
| Beeswax | 0.1 |

### Example 2—Topical Balm

A mixture of the following excipients is prepared in the form of a topical balm.

| | Amount |
|---|---|
| Component | (wt. %) |
| Manuka oil | 0.05-1.0 |
| Black seed oil | 0.05-1.0 |
| Ethylhexyl palmitate | 0.5-5.0 |
| Kahl VegoJelly 7036Plus (carrier, mixture of Ricinus | 78-99.4 |
| Communis seed oil, Rhus Verniciflua peel wax, Rhus | |
| Succedanea fruit wax, ascorbyl palmitate, and tocopherol). | |
| Jojoba oil | 0-5.0 |
| Beeswax | 0-5.0 |
| Vitamin a | 0-5.0 |
| Total | 100 |

### Example 3—Topical Blemish Gel

A mixture of the following excipients is prepared in the form of a topical gel for the treatment of skin blemishes including abrasions, acne, or other irritations.

| | Amount |
|---|---|
| Component | (wt. %) |
| Manuka oil | 0.01-1.0 |
| Black seed oil | >0-1.0 |
| Aqua (water) | 75-balance |
| Glycerin | 1-5 |
| Xantham Gum | 0.1-1 |
| Benzyl alcohol | 0.1-1 |
| Polysorbate 20 | 0.1-1 |
| Citrius limon peel oil | >0-0.1 |
| Retinyl palmitate | >0-0.1 |
| Dehydroacetic acid | >0-0.1 |
| Calendula officinalis flower extract | >0-0.1 |
| BHT (antioxidant) | >0-0.1 |
| Limonene | >0-0.1 |
| Citral | >0-0.1 |
| Linalool | >0-0.1 |
| Total | 100 |

The disclosed manuka oil and black seed oil based blemish gel showed success as an acne spot treatment to prevent bacteria, makeup, and environmental debris from interfering with the skin's natural recovery process.

### Example 4—Minimum Inhibitory Concentration (MIC)

The MIC test evaluates the lowest concentration of a test substance that prevents growth of the microbe. This is a measure of the antibiotic activity of the test sample. The disclosed topical compositions may be tested for MIC using publicly available testing criteria. For example, test samples of the desired active compound can be prepared and serially diluted to give 11 or more different concentrations covering, for example, a 1000-fold range. For example, the test samples may include 50%-0.1% of a stock test sample (e.g., stock samples containing 1.32% Manuka oil, 0.33% black seed oil) that are combined with select bacteria and fungi and incubated over a prescribe period of time. The MIC is useful to determine the lowest concentration of each test sample (mixture of active materials) that inhibits the growth of the microbe as determined by measurement of OD₆₅₀ₙₘ using a Versa Max 96 well plate reader. Measurement of cell concentration can be made at several time points.

The methodology for growing the different micro-organisms available for evaluation in the MIC studies may be provided by the CLSI standards, Methods for Dilution Antimicrobial Susceptibility Tests for Bacteria that Grow Aerobically, Approved Standard, Ninth Edition, M07-A9, Vol. 32 No 2, January 2012; Methods for Antimicrobial Susceptibility Testing for Anaerobic Bacteria. Approved Standard, Ninth Edition, M11-9, January 2018; and Performance Standards for Antifungal Susceptibility Testing of Filamentous Fungi. First Edition, M61, November 2017.

Microorganisms that may be tested include, but are not limited to, Acinetobacter bawnanni; Candida albicans; Colynebacteriwn diphtheria; Cutibacterium acnes; Enterococcus faecium; Escherichia coli; Proteus vulgans; Pseudomonas aeruginosa; Staphylococcus aureus; Staphylococcus aureus (MRSA); Stapllylococcus capilis; Stapllylococcus epidermidis; Stapllylococcus pseudinlermedius; Streptococcus pyogenes; Tricllophyton menlagrophytes; and Trichophywn rubrum. The disclosed manuka oil and black seed combination show promising initial results and may be viable for MIC testing against inhibiting growth of one or more of the above microorganisms.

Known antibiotic samples can also be tested as a comparative sample and control. Such antioxidants can include, but are not limited to Amoxicillin, Cefalotin, Colistin, Erythromycin, Itraconazole, Metronidazole, and Penicillin.

What is claimed is:

1. A topical composition for treatment of a skin condition comprising a mixture of:
Leptospermum scoparium oil (manuka oil);
Nigella sativa seed oil (black seed oil); and
a carrier composition.

2. The topical composition of claim 1, further comprising ethylhexyl palmitate.

3. The topical composition of claim 1, wherein the carrier composition comprises one or more natural waxes, antioxidants, emollients, moisturizers, or combinations thereof.

4. The topical composition of claim 3, wherein the carrier composition comprises one or more of beeswax, Rhus succedanea fruit wax, Rhus verniciflua peel wax, Ricinus communis seed oil (castor oil), Simmondsia chinensis seed oil (jojoba seed oil), ascorbyl palmitate, and tocopherol.

5. The topical composition of claim 4, wherein the carrier composition comprises beeswax, Rhus succedanea fruit wax, Rhus verniciflua peel wax, Ricinus communis seed oil (castor oil), Simmondsia chinensis seed oil (jojoba seed oil), ascorbyl palmitate, and tocopherol.

6. The topical composition of claim 1, wherein the manuka oil comprises at least 5 weight percent (wt. %) β-triketones.

7. The topical composition of claim 1, wherein the topical composition comprises a ratio of manuka oil to black seed oil of about 1:1 to about 10:1 by weight.

8. The topical composition of claim 7, wherein the ratio of manuka oil to black seed oil is about 4:1.

9. The topical composition of claim 2, wherein the topical composition comprises:
about 0.05 weight percent (wt. %) to about 1 wt. % manuka oil based on a total weight of the topical composition;
about 0.05 wt. % to about 1 wt. % black seed oil based on the total weight of the topical composition; and
about 0.5 wt. % to about 5 wt. % ethylhexyl palmitate based on the total weight of the topical composition.

10. The topical composition of claim 2, wherein the topical composition comprises:
about 0.4 wt. % manuka oil based on a total weight of the topical composition;
about 0.1 wt. % black seed oil based on the total weight of the topical composition; and
about 1 wt. % ethylhexyl palmitate based on the total weight of the topical composition.

11. The topical composition of claim 2, wherein the topical composition is formulated as a skin balm.

12. The topical composition of claim 1, wherein the topical composition is for use in inhibiting microbial growth on skin, treating a skin abrasion or laceration, or treating acne.

13. A method for treating a skin condition comprising:
applying a topical composition to an area containing a skin condition, the topical composition comprising:
Leptospermum scoparium oil (manuka oil);
Nigella sativa seed oil (black seed oil); and
a carrier composition, wherein the manuka oil, the black seed oil, and the ethylhexyl palmitate are mixed in the carrier composition.

14. The method of claim 13, wherein the topical composition further comprises ethylhexyl palmitate.

15. The method of claim 13, wherein the topical composition is in the form of a topical balm or topical gel.

16. A method for manufacturing a topical composition used to treat a skin condition comprising mixing Leptospermum scoparium oil (manuka oil) and Nigella sativa seed oil (black seed oil) into a carrier composition.

17. The method of claim 16, further comprising mixing ethylhexyl palmitate into the carrier composition and forming a topical balm.
